(19) United States
(12) Patent Application Publication
(10) Pub. No.: US 2021/0321910 A1
(43) Pub. Date: Oct. 21, 2021

(54) System and Method for Detecting Auditory Biomarkers

(71) Applicants: Takeichi Kanzaki CABRERA, Palmer, MA (US); Stephen DONOGHUE, Palmer, MA (US); Alec MIAN, Palmer, MA (US); Curelator, Inc., Palmer, MA (US)

(72) Inventors: Takeichi Kanzaki Cabrera, Palmer, MA (US); Stephen Donoghue, Palmer, MA (US); Alec Mian, Palmer, MA (US)

(21) Appl. No.: 17/312,563

(22) PCT Filed: Dec. 12, 2019
(86) PCT No.: PCT/US2019/066071

§ 371 Date: Jun. 10, 2021

Related U.S. Application Data
(60) Provisional application No. 62/778,623, filed on Dec. 12, 2018.

(51) Int. Cl. A61B 5/12; A61B 5/00

(52) U.S. Cl. CPC A61B 5/123; A61B 5/7275; A61B 5/7415

(57) ABSTRACT

The disclosed systems and methods include providing a plurality of sounds at varying intensity levels, and receiving an input indicative of a perceived volume level of the plurality of sounds as perceived by a user. The disclosed systems and methods include determining, based on the received input, a plurality of user volume levels that indicate a hearing sensitivity of the user. The disclosed systems and methods include providing a background sound, and concurrently while providing the background sound, providing a stimulus sound. The disclosed systems and methods include receiving an indication that the user perceived the stimulus sound. The disclosed systems and methods include determining a change in the hearing sensitivity of the user based on receiving the indication that the user perceived the stimulus sound, and predicting an onset of a migraine attack of the user based on determining the change in the hearing sensitivity of the user.

### CROSS-REFERENCE TO RELATED APPLICATIONS

This application claims priority to U.S. provisional application 62/778,623, filed on Dec. 12, 2018, the entire content of which is incorporated herein by reference.

### BACKGROUND

Some current techniques for patients to manage disease symptoms include keeping a written record of times when disease symptoms occur as well as times when the patient engages in potential triggering (and perhaps mitigating) activities. Other current techniques can include a patient keeping an electronic diary of disease symptoms, disease triggering/mitigating activities, along with perhaps other disease monitoring and management related data, perhaps by way of an application. A type of data that can be considered in tracking disease symptoms relate to sensory thresholds of patients, such as threshold levels associated with sounds heard by patients.

### SUMMARY

Some migraine patients report difficulty with understanding speech and/or discerning other sounds in the days and hours leading up to (and sometimes during) a migraine episode. For migraine patients who experience this short-term change in hearing capability, this change provides a biomarker for migraine because it is a measurable indicator of biological changes that occur in the days and hours leading up to a migraine episode. The ability to detect biomarkers for migraine is useful not only to predict onset of a migraine episode and give migraine patients an opportunity for early therapeutic intervention to prevent or at least ameliorate the effects of a migraine headache, but also for clinical testing as well as drug discovery and development.

This application discloses systems and methods for detecting auditory migraine biomarkers in migraine patients. A patient (more generally referred to as “a user”), can interact with a software applicant (perhaps a healthcare management application) for purposes of detecting auditory biomarkers for a migraine. In certain embodiments and applications of the description, set forth herein the user is also a patient or an individual suffering from a disease, disorder, or condition, particularly a migraine. Accordingly, to the extent the term “patient” is referred to herein, it should be understood that the operations described can similarly be applied to a user (e.g., an individual that is not receiving acute treatment in association with the software application). Although these systems and methods are directed to changes to hearing capabilities that predict onset of a migraine attack, the disclosed systems and methods described herein can also be applicable to other diseases for which changes in hearing capability can indicate onset and/or presence of such other diseases and/or predict the occurrence of episodes of, or periods of worsening of symptoms. For example, many aspects of these systems and methods can be applicable to detecting biomarkers in other neurological diseases with episodic attacks, such as epilepsy, depression, and others. Within examples described herein, a user may be referred to as “perceiving” a sound signal. The term “perceived” is intended to encompass the human senses, particularly hearing. Thus, within the examples provided herein, when a user and/or patient is described as “hearing” a sound, this can be understood as the user and/or patient perceiving the sound.

In a first aspect, a method is provided comprising steps of: providing, by an audio output device, a plurality of sounds at varying intensity levels. The method includes receiving, via a user interface, an input indicative of a perceived volume level of the plurality of sounds as perceived by a user. The method includes determining, based on the received input, a plurality of user volume levels. The plurality of user volume levels includes (i) a threshold audible volume for the user, (ii) a comfortable volume level for the user, and (iii) a maximum comfort volume level for the user. The plurality of user volume levels indicates a hearing sensitivity of the user. The method includes providing, by the audio output device, a background sound. The method includes, concurrently while providing the background sound, providing, by the audio output device, a stimulus sound. The method includes receiving an indication, via the user interface, from the user that the user perceived the stimulus sound. The method includes determining a change in the hearing sensitivity of the user based on receiving the indication that the user perceived the stimulus sound. The method includes predicting an onset of a migraine attack of the user based on determining the change in the hearing sensitivity of the user.

In a second aspect, a non-transitory computer readable medium is provided. The non-transitory computer readable medium has computer-executable program code stored thereon that, when executed by one or more processors, causes performance of one or more functions. The functions include providing, by an audio output device, a plurality of sounds at varying intensity levels. The functions include receiving, via a user interface, an input indicative of a perceived volume level of the plurality of sounds as perceived by a user. The functions include determining, based on the received input, a plurality of user volume levels. The plurality of user volume levels includes (i) a threshold audible volume for the user, (ii) a comfortable volume level for the user, and (iii) a maximum comfort volume level for the user. The plurality of user volume levels indicate a hearing sensitivity of the user. The functions include providing, by the audio output device, a background sound. The functions include, concurrently while providing the background sound, providing, by the audio output device, a stimulus sound. The functions include receiving an indication, via the user interface, from the user that the user perceived the stimulus sound. The functions include determining a change in the hearing sensitivity of the user based on receiving the indication that the user perceived the stimulus sound. The functions include predicting an onset of a migraine attack of the user based on determining the change in the hearing sensitivity of the user.

In a third aspect, a system is provided that includes a computing device. The computing device is configured to perform one or more functions. The functions include providing, by an audio output device, a plurality of sounds at varying intensity levels. The functions include receiving, via a user interface, an input indicative of a perceived volume level of the plurality of sounds as perceived by a user. The functions include determining, based on the received input, a plurality of user volume levels. The plurality of user volume levels includes (i) a threshold audible volume for the user, (ii) a comfortable volume level for the user, and (iii) a maximum comfort volume level for the user. The plurality of user volume levels indicate a hearing sensitivity of the user. The functions include providing, by the audio output device, a background sound. The functions include, concurrently while providing the background sound, providing, by the audio output device, a stimulus sound. The functions include receiving an indication, via the user interface, from the user that the user perceived the stimulus sound. The functions include determining a change in the hearing sensitivity of the user based on receiving the indication that the user perceived the stimulus sound. The functions include predicting an onset of a migraine attack of the user based on determining the change in the hearing sensitivity of the user.

In a fourth aspect a system is provided. The system includes means for performing one or more functions. The functions include providing, by an audio output device, a plurality of sounds at varying intensity levels. The functions include receiving, via a user interface, an input indicative of a perceived volume level of the plurality of sounds as perceived by a user. The functions include determining, based on the received input, a plurality of user volume levels. The plurality of user volume levels includes (i) a threshold audible volume for the user, (ii) a comfortable volume level for the user, and (iii) a maximum comfort volume level for the user. The plurality of user volume levels indicate a hearing sensitivity of the user. The functions include providing, by the audio output device, a background sound. The functions include, concurrently while providing the background sound, providing, by the audio output device, a stimulus sound. The functions include receiving an indication, via the user interface, from the user that the user perceived the stimulus sound. The functions include determining a change in the hearing sensitivity of the user based on receiving the indication that the user perceived the stimulus sound. The functions include predicting an onset of a migraine attack of the user based on determining the change in the hearing sensitivity of the user.

Some embodiments of the systems and methods disclosed herein include an auditory test implemented with a smartphone or other computing device. In operation, a migraine patient listens to auditory signals generated by a software application running on the smartphone via headphones connected to the smartphone. Such software applications can vary aspects of the auditory signals in a specific manner to detect migraine biomarkers.

In some embodiments, a software application on the patient's smartphone first generates a background sound (such as but not limited to Gaussian white noise) at a particular volume level. This background sound is sometimes referred to herein as “the background.” Then, while playing the background, the software application generates an audio stimulus sound (such as but not limited to a discreet tone, sound, or word) at varying volume levels and at varying intervals in monaural and/or in stereo via the right and left channels. This audio stimulus sound is sometimes referred to herein as “the stimulus.” In some embodiments, a volume level of the stimulus is initially set low, but the system increases the volume level of the stimulus over time as described further herein. In some embodiments, the software application generates a user interface screen comprising a “left” and “right” button, and instructs the patient to tap the button (left or right) corresponding to the ear (left or right) via which the patient hears the stimulus.

In some embodiments, the background comprises one or more of (i) a set of static or dynamic, specific frequencies at random or specific intensities, (ii) a set of static or dynamic, random frequencies at random or specific intensities, (iii) a particular type of random noise, such as but not limited to white, pink, brown, blue, violet, grey, or other type of random noise at a random or a specific intensity, (iv) a single, static or dynamic, specific tone at a random or specific intensity, (v) a single, static or dynamic, random tone at a random or specific intensity, (vi) pre-recorded background noise, such as but not limited to a recording of a coffee shop, café, traffic, train, rain, waves, etc., at a random or specific intensity, and/or (vii) any combination of the foregoing. In some embodiments, background intensity can vary over time. In some embodiments, background intensity varies as a function of time between about 0 dB and 120 dB sound pressure level (SPL). In some embodiments, background intensity remains constant for a set of auditory biomarker detection tests performed over a few hours or a few days, but the background intensity can be changed from time to time to provide better data on the degree to which patients are able to discern the stimulus from the background when the background is set to higher or lower intensity (volume) levels.

In some embodiments, the background is set at a patient's maximum comfort level, i.e., at an intensity (volume) level that is just before the patient deems the background to be uncomfortable. In some embodiments, the background is set just above a patient's threshold level, i.e., at an intensity (volume) level that is just high enough for the patient to hear. In some embodiments, the background is set within a patient's comfort level, i.e., at an intensity (volume) level that is higher than the patient's threshold level but below the patient's maximum comfort level. Some embodiments will not include or otherwise use a background. Regardless of the absence, presence, and/or intensity level of the background, it is desirable in some embodiments to use the same (or at least substantially the same) volume levels (i.e., the patient's maximum comfort level, the patient's threshold level, and the patient's comfort level/range) for a particular patient at least for a set of multiple auditory biomarker detection tests performed over the course of a few hours or a few days.

In some embodiments, a stimulus comprises one or more of (i) a patient's name, (ii) randomized words, (iii) randomized tones of varying frequency and intensity, (iv) other words and/or tones, and/or (v) any combination of the foregoing. In some embodiments, regardless of the stimulus content, the frequency and/or intensity of the stimulus can vary over time. In some embodiments, the stimulus intensity can be increased or decreased during a testing session within defined increments. For example, in some embodiments, the stimulus intensity is increased and/or decreased in increments of between about 1-6 dB.

In some embodiments, a stimulus can be continuous or pulsed within a time interval. For some embodiments, the stimulus is pulsed over a fixed time interval. For some embodiments, the stimulus is pulsed over a time interval that varies over time randomly or according to a specific pattern. For example, in some embodiments, the time interval between successive stimulus sounds varies between about 1-10 seconds. In some embodiments employing a pulsed stimulus, the stimulus comprises between about 3 and 1000 pulses delivered during a single time interval of between about 1-10 seconds. In some embodiments, a single testing session comprising multiple iterations of an auditory biomarker detection test lasts between about 2-15 minutes.

In some embodiments, one or both of a stimulus and/or a background can comprise customized sound tracks. Some example sound tracks that can be used by a system as a stimulus and/or background include: (i) a “Sirens of Odysseus” sound track, where the background comprises sounds of crashing waves and howling wind that fluctuate between a narrow range of intensities, and where the stimulus comprises a patient's name called intermittently at varying intensities (loudness) and/or frequencies; (ii) a “Lost in the Jungle” sound track, where a background comprises sounds of leaves rustling, jungle noises, running streams of water, and/or other jungle sounds that fluctuate within a narrow range of intensities, and where the stimulus comprises the name “Tarzan” called intermittently at varying intensities (loudness) and/or frequencies; and/or (iii) a “Rain Storm” sound track, where background comprises a steady sound of falling rain and wind, and where the stimulus is a user's name (or perhaps some other word) called intermittently at varying intensities (loudness) and/or frequencies. In operation, because the biomarker detection system is aimed at identifying temporary changes in hearing capability that occur within days and hours leading up to a migraine attack, the above-described example sound tracks and other sound tracks are effective for analyzing within person variation over periods of a few days or perhaps a few hours. In some embodiments, the biomarker detection system can identify and track variations in the temporary changes to a patient's hearing capabilities over many weeks, months, or years.

To prevent a patient from learning how to discern the stimulus sound from the background sound and anticipating the system's playback of the stimulus sound based on repetition, in some embodiments, the content, intensity, frequency, and/or pulse rate of the stimulus sound and/or the background sound is advantageously varied within one or both of (i) a single testing session where the system performs the migraine biomarker detection procedure and (ii) different testing sessions where the system varies the background sound and/or the stimulus sound attributes used for different testing sessions. In some embodiments, the extent and degree to which the system varies one or more of the content, intensity, and/or duration of the background signal and/or the stimulus sound varies based on one or more of a patient's age, sex, or other distinguishing characteristics between patients.

In some embodiments, systems and methods begin for a patient by setting an initial volume level within the patient's personal hearing range. Some embodiments include determining an initial volume level by playing one or more test sounds to the patient (such as but not limited to via headphones connected to the patient's smartphone) and receiving one or more confirmations (such as but not limited to, via the user interface) from the patient that he or she heard (or perhaps did not hear) the test sounds. Some embodiments can additionally include determining a hearing range for a patient by playing a plurality of test sounds by providing, by an audio output device, a plurality of sounds at varying intensity levels (e.g., via headphones connected to the patient's smartphone) and receiving a plurality of confirmations (e.g., via the user interface) from the patient of both (i) the lowest volume test sound that the patient heard and (ii) the loudest volume test sound that the patient heard before the volume became too uncomfortably loud for the patient. In some embodiments, the system and method includes storing an individual patient's hearing range, defined by the patient's threshold volume level and the patient's maximum comfort level.

In some embodiments, the system next plays a first set of stimulus sounds (with or without background) at random intensity levels (volumes) that range in intensity from below the patient's threshold volume level to up to the patient's maximum comfort level. The system tracks whether the patient heard each stimulus in the first set of stimulus sounds based on whether the system received a confirmation (e.g., via the user interface) that the patient heard the stimulus. Then, in some embodiments, the system uses that first set of confirmations received from the patient in response to playing the first set of stimulus sounds to play a second set of stimulus sounds. The second set of stimulus sounds have intensity levels that are within a narrower range of intensities compared to the first set of stimulus sounds, where this narrower range of intensities ranges from just below the lowest-detected intensity (volume) confirmed by the patient in response to the first set of stimulus sound to a volume level just above the volume of the first-detected stimulus sound in the first set of stimulus sounds. In addition to having a narrower range of intensities as compared to the first set of stimulus sounds, intensity level differences between individual stimulus sounds in the second set of stimulus sounds is also smaller than intensity level differences between individual stimulus sounds in the first set of stimulus sounds. Thus, in some embodiments, the first set of stimulus sounds can function to identify a narrower range in which to conduct a more-focused testing session with the second set of stimulus sounds.

Testing as described herein is directed to obtaining as precise as possible assessment of a patient's hearing sensitivity and/or ability to discern the stimulus from the background each day (or perhaps a few times a day) and measure changes over time for an individual patient. In some embodiments, results from an individual patient as well as from sets of individual patients can be compiled and correlated with instances of migraine attacks for the patient and/or the sets of patients to help identify auditory biomarkers for each individual patient and/or perhaps for a group of patients sharing similar patient characteristics. Once a reliable auditory biomarker (e.g., combination of stimulus and background sounds at particular intensities, frequencies, durations, and so on) has been identified for an individual patient, the systems and methods disclosed and described herein can focus on using the identified reliable auditory biomarker for that patient, which would provide more reliable migraine prediction for that patient.

Because noise sensitivity is also a premonitory symptom for some migraine patients, some embodiments can additionally include a loudness/discomfort test that includes receiving one or more indications from a patient (e.g., via the graphical user interface) for one or more of (i) when the patient can discriminate between a stimulus sound and a background sound and/or (ii) when a stimulus sound, individually or in combination with a background sound, becomes uncomfortably loud or irritating or bothersome for the patient.

In some embodiments, the systems and methods disclosed and described herein can be used on their own or perhaps in combination with other similar sensory discrimination tests (e.g., visual, tactile, etc.) and the presence of other premonitory symptoms and/or potential risk factors using various algorithms, including machine learning and/or artificial intelligence techniques. In some instances, using the disclosed auditory biomarker tests in combination with one or more other sensory discrimination tests can help confirm onset of attacks and help reduce the likelihood of false positive results.

For example, in some embodiments, the systems and methods disclosed and described herein can be used in combination with systems and methods disclosed and described in one or both of (1) U.S. application Ser. No. 15/502,087 titled “Chronic Disease Discovery and Management System,” filed on Feb. 6, 2017, and which claims priority to (a) PCT application PCT/US15/43945 titled “Chronic Disease Discovery and Management System,” filed on Aug. 6, 2015, (b) U.S. provisional application 62/034,408 titled “Disease Symptom Trigger Map,” filed on Aug. 7, 2014; (c) U.S. provisional application 62/120,534 titled “Chronic Disease Management System,” filed on Feb. 25, 2015; (d) U.S. provisional application 62/139,291 titled “Chronic Disease Discovery and Management System,” filed on Mar. 27, 2015, (e) U.S. provisional application 62/148,130 titled “Chronic Disease Discovery and Management System,” filed on Apr. 15, 2015; and (f) U.S. provisional application 62/172,594 titled “Chronic Disease Discovery and Management System,” filed on Jun. 8, 2015; and/or (2) PCT application PCT/US14/13894 titled “Methods and Systems for Determining a Correlation Between Patient Actions and Symptoms of a Disease,” which claims priority to (a) U.S. provisional application 61/860,893 titled “Methods and Systems for Determining a Correlation Between Patient Actions and Symptoms of a Disease,” filed on Jul. 31, 2013, (b) U.S. provisional application 61/762,033 titled “Methods and Systems for Determining a Correlation Between Patient Actions and Symptoms of a Disease,” filed on Feb. 7, 2013; and (c) U.S. provisional application 61/759,231 titled “Methods and Systems for Determining a Correlation Between Patient Actions and Symptoms of a Disease,” filed Jan. 31, 2013. All of the above-listed applications are owned by Curelator, Inc., and this application incorporates the entire contents of all of the above-listed applications by reference.

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that aspects of the present disclosure, as generally described herein, can be arranged, substituted, combined, separated, and designed in a wide variety of different configurations, all of which are explicitly contemplated herein.

### BRIEF DESCRIPTION OF THE FIGURES

FIG. 1 shows a first method employed in various example embodiments of the systems and methods for detecting auditory biomarkers disclosed herein.

FIG. 2 shows a second method employed in various example embodiments of the systems and methods for detecting auditory biomarkers disclosed herein.

FIG. 3 shows a third method employed in various example embodiments of the systems and methods for detecting auditory biomarkers disclosed herein.

FIG. 4 shows a fourth method employed in various example embodiments of the systems and methods for detecting auditory biomarkers disclosed herein.

FIG. 5 shows an example computing device configured to execute the features and functions of the auditory biomarker detection methods disclosed and described herein.

### DETAILED DESCRIPTION OF EXAMPLE EMBODIMENTS SHOWN IN THE FIGURES

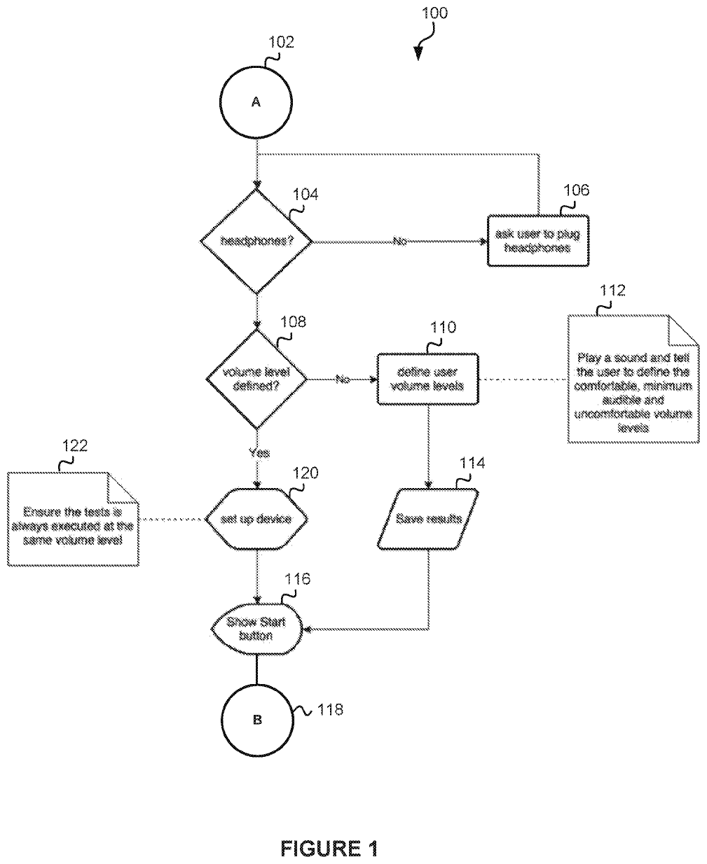

FIGS. 1-4 illustrate four methods 100, 200, 300, and 400. In some embodiments, the systems and methods disclosed and described herein implement or otherwise include features and functions in one or more (or all) of the four methods. Segmentation of these features and functions into four methods shown here is solely for convenience and ease of illustration. Some embodiments can include more or fewer features and functions, and these features and functions can be organized and arranged into more or fewer methods or perhaps not divided into multiple methods at all.

FIG. 1 illustrates a first method 100 employed in various example embodiments of the systems and methods for detecting auditory biomarkers disclosed herein.

Method 100 begins at starting point 102, where a smartphone or other computing device (referred to herein generically as a computing device) launches a software application for detecting auditory migraine biomarkers. In some embodiments, the computing device launches the application in response to a request received (e.g., via a user interface) from a patient, e.g., a smartphone launches the application in response to the patient selecting the application via the user interface.

Next, method 100 advances to block 104, where the application determines whether headphones are connected to the computing device. In operation, headphones can be connected to the computing device via any wired or wireless connection now known or later developed, e.g., a standard analog audio jack, Bluetooth™, Lightning™, or other type of connection. If headphones are not connected to the computing device, method 100 advances to block 106, where the application prompts the patient to connect the headphones to the computing device. If at block 104, the application determines that headphones are connected to the computing device, then in some embodiments, the application additionally prompts the patient to confirm that the “right” earphone is in/over the patient's right ear and that the “left” earphone is in/over the patient's left ear by playing confirmatory sounds via one or both earphones.

For example, the application can play a phrase via the “left” earphone such as, “Please be sure this earphone is in your left ear” while displaying a prompt via the user interface screen for the patient to confirm that he or she is wearing the earphones correctly. Alternatively, the application can play a sound or phrase via one earphone such as, “Do you hear this sound in your right ear or in your left ear?” and display a prompt via the user interface screen for the patient to confirm which ear he or she heard the sound. And depending on which ear the patient confirmed hearing the sound, the application can adjust how it plays sound throughout the remainder of the test. For example, if the application played the phrase, “Do you hear this sound in your right ear or in your left ear?” via the right earphone, and if the patient confirmed hearing the sound in his or her right ear, then the application has confirmed that the patient is wearing the earphones correctly and the test can proceed without modification to how the application plays sounds via the right and left channels. But if the application played the phrase, “Do you hear this sound in your right ear or in your left ear?” via the right earphone, and if the patient confirmed hearing the sound in his or her left ear, then the application has confirmed that the patient is wearing the earphones in the wrong ears, and the application can either instruct the patient to wear the earphones correctly or the application can reverse the ears for the test and play the right channel via the left earphone and left channel via the right earphone to account for the patient wearing the earphones backwards.

After receiving confirmation that the patient is wearing the earphones correctly, method 100 advances to block 108.

At block 108, method 100 determines whether one or more volume levels have been defined for the patient. In some embodiments, at least one volume level is a level that is higher than the patient's threshold level and below the patient's maximum comfort level.

If the patient's volume levels have not been defined, method 100 advances to block 110, where patient volume levels are defined. In some embodiments, defining the patient volume levels includes playing a plurality of sounds, by an audio output device (e.g., via headphones connected to the patient's smartphone), at varying intensity levels and asking the patient to define one or more of a comfortable volume, a minimum audible (or threshold) volume, and/or an uncomfortable (or perhaps maximum comfort) volume level, as summarized in comment block 112 of method 100. Because a patient's hearing ability and sensitivity can vary between his or her left and right ears, some embodiments include defining a comfortable volume, threshold volume, and/or maximum comfort volume level for each ear independently. After defining the patient's volume levels in block 110, method 100 advances to block 114, where the application stores the patient's determined volume levels. In some embodiments, block 114 can additionally include configuring the application to perform an auditory biomarker test based on the patient's determined volume levels. After saving the patient's determined volume levels at block 114 (and perhaps also configuring the application with the patient's determined volume levels), method 100 advances to block 116, where the application displays a “Start” (or similar) icon via a graphical user interface.

If, at block 108, the application determines that the patient's volume levels have already been defined, then method advances to block 120, where the application is configured to perform an auditory biomarker test based on the patient's pre-defined volume levels. In some embodiments, it is desirable to ensure the application uses the same (or substantially the same) patient volume levels for individual auditory biomarker detection tests within a defined time period, as summarized in comment block 122. For example, in some embodiments, it is desirable for the application to use the same (or substantially the same) patient volume levels (threshold, maximum comfort, and corresponding comfort range) for each of a plurality of auditory biomarker detection tests performed over the course of a few hours to a few days so that slight changes in the patient's ability to discern the stimulus from the background over the course of a few hours to a few days can be measured and tracked. As previously stated, in some embodiments, the application can identify and track variations in the temporary changes to a patient's hearing capabilities over many weeks, months, or years.

After the application is configured to perform the auditory biomarker test at block 120, method 100 advances to block 116, where the application displays a “Star” (or similar) icon via a graphical user interface After displaying the “Start” (or similar) icon at block 116, method 100 ends at point 118, which is also the starting point for method 200.

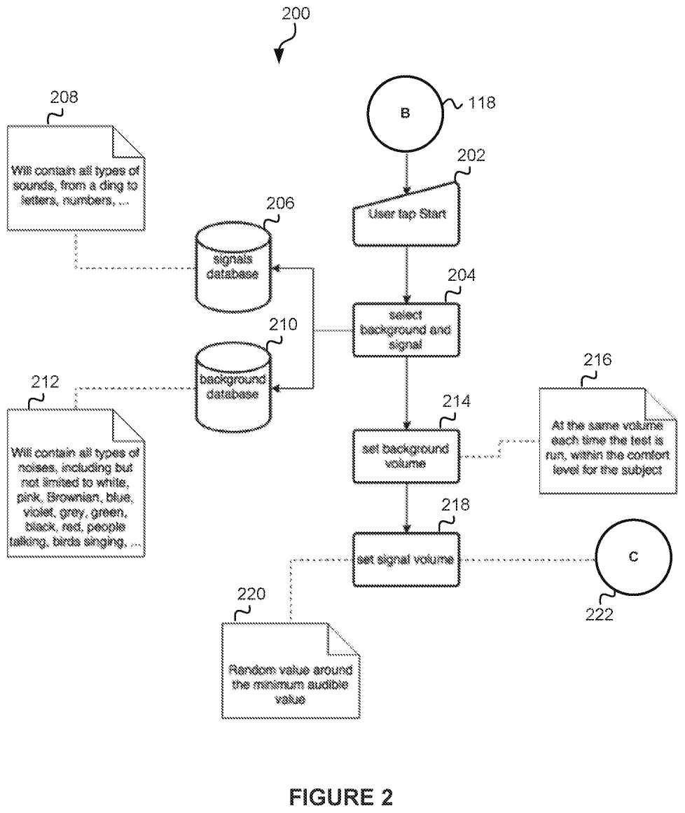

FIG. 2 shows a second method 200 employed in various example embodiments of the systems and methods for detecting auditory biomarkers disclosed herein. Method 200 begins at starting point 118, where the application waits to receive a patient input to start method 200. In some embodiments, at block 118, the application can provide a patient with instructions for configuring one or more parameters that the application will use when performing the auditory biomarker detection test.

At block 202, the application receives an input from the patient to configure the application for performing the auditory biomarker detection procedure. For example, in some embodiments, the application receives a command to start the application configuration procedure via a graphical user interface.

After receiving the command to start the application configuration procedure, method 200 advances to block 204, where the application receives inputs (e.g., via the graphical user interface) to select one or more background and/or stimulus sounds. In some embodiments, the one or more background and/or stimulus sounds are selected from a background database 210 and a stimulus database 206, respectively. In some embodiments, the background database 210 includes many different background sounds for use as the background, including but not limited to white noise, pink noise, Brownian noise, blue noise, violet noise, grey noise, green noise, black noise, red noise, talking people, singing birds, any of the other background sounds disclosed herein, and/or any other background sound now known or later developed that is suitable for use as a background sound for an auditory biomarker detection test, as summarized in comment block 212 of method 200. Similarly, in some embodiments, the stimulus database 206 includes many different stimulus sounds for use as the stimulus, including but not limited to various types of beeps, dings, words, phrases, numbers, letters, names, animal sounds, and/or any other type of sound now known or later developed that is suitable for use as a stimulus sound for an auditory biomarker detection test, as summarized in comment block 208.

After the background and/or stimulus sounds have been selected at block 204, method 200 advances to block 214, where the background volume is set. In some embodiments, the application sets the background volume at the same level each time the auditory biomarker detection test is performed. In some embodiments, the background volume is based at least in part on the patient volume levels (threshold, maximum comfort, and corresponding comfort range) determined in method 100, e.g., determined at block 110 and/or retrieved from memory.

After setting the background volume at block 214, method 200 advances to block 218, where the application sets one or more volume levels for one or more corresponding stimulus sounds. In some embodiments, the stimulus sound volume is based at least in part on patient volume levels (threshold, maximum comfort, and corresponding comfort range) determined in method 100, e.g., determined at block 110 and/or retrieved from memory. In some embodiments, the application sets each of the one or more volume levels for each of the one or more stimulus sounds at different random volume levels that are around (e.g., within about +/−1 dB to 6 dB) of a patient's threshold volume level for each iteration of the auditory biomarker detection test during a testing session.

After setting the one or more stimulus sound volume levels at block 218, method 200 ends at point 222, which is also the starting point for method 300.

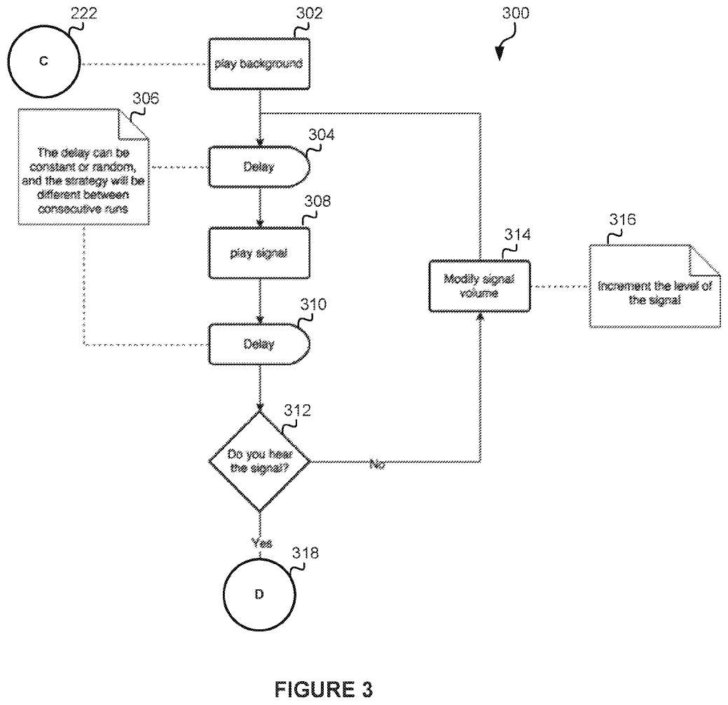

FIG. 3 shows a third method 300 employed in various embodiments of the systems and methods for detecting auditory biomarkers disclosed herein. Method 300 begins at starting point 222, where the application waits to receive a patient input to start method 200. In some embodiments, at block 222, the application can provide the patient with instructions for performing the auditory biomarker detection test.

Next, method 300 advances to block 302, where the application plays a background sound. In some embodiments, the application plays the background sound selected at block 204 of method 200 at the background volume selected or set at block 214 of method 200.

While continuing to play the background sound, method 300 advances to block 304, where a first delay is implemented before advancing to block 308, where the application plays a stimulus sound. In some embodiments, at block 308, the application plays the stimulus sound selected at block 204 of method 200 at a first one of the one or more stimulus sound volume levels set at block 218 of method 200. In some embodiments, the application plays the stimulus sound at block 308 for a fixed or random duration of time.

After playing the stimulus sound at block 308, and while continuing to play the background sound, method 300 advances to block 310, where a second delay is implemented before advancing to block 312. In some embodiments, one or both of the first delay implemented at block 304 and the second delay implemented at block 310 can be a fixed or random duration of time. Similarly, the first delay implemented at block 304 and the second delay implemented at block 310 can be the same duration of time or different durations of time that are fixed or random, as summarized in comment block 306.

After implementing the second delay at block 310, and while continuing to the play the background sound, method 300 advances to block 312, where the application asks the patient whether the patient heard the stimulus sound. For example, the application can generate and display a prompt on the user interface with “Yes” and “No” (or similar) icons for the patient to select based on whether the patient heard the stimulus sound.

If the application receives an indication from the patient that the patient did not hear the stimulus sound (e.g., a “No” input via the graphical user interface), then method 300 advances to block 314, where the application modifies the volume of the stimulus sound, where modifying the volume of the stimulus sound includes incrementing the volume, as summarized in comment block 316. In some embodiments, modifying the volume of the stimulus sound at block 314 additionally or alternatively includes decrementing the volume level of the stimulus sound. In some embodiments, modifying the volume of the stimulus sound additionally or alternatively includes setting the volume of the stimulus sound to a second one of the one or more stimulus sound volume levels set at block 218 of method 200.

After modifying the volume of the stimulus sound at block 314, and while continuing to play the background sound, method 300 returns to block 304, where the application implements a first delay (which could be the same or a different duration as when the application previously implemented the first delay at block 304). And after expiration of the first delay at block 304, method 300 advances again to block 308, where the application plays the stimulus sound again, but at the modified volume level set at block 314. In some embodiments, the application plays the stimulus sound at block 308 again for a fixed or random duration of time, which can be the same or a different duration than when the application previously played the stimulus sound at block 308.

After playing the stimulus sound at block 308, method 300 again advances to block 310, where the application implements the second delay (which could be the same or a different duration as when the application previously implemented the second delay at block 310).

After expiration of the second delay at block 310, method 300 advances again to block 312, where the application again asks the patient whether the patient heard the stimulus sound. For example, the application can again generate and display a prompt on the user interface with “Yes” and “No” (or similar) icons for the patient to select based on whether the patient heard the stimulus sound.

If the application receives an indication from the patient that the patient again did not hear the stimulus sound (e.g., a “No” input via the graphical user interface), then method 300 returns to block 314, and method 300 continues in a loop-wise, iterative fashion traversing blocks 304, 308, 310, and 312 until either: (i) the application receives an indication from the patient that the patient heard the stimulus sound (e.g., a “Yes” input via the graphical user interface) or (ii) the application receives a certain quantity of “No” indications from the patient that the patient did not hear the stimulus sound (e.g., a “No” input via the graphical user interface). For example, in some embodiments, if after a few iterations (e.g., between about 7-15) the patient still cannot hear the stimulus sound, then method 300 will stop and perhaps instruct the patient to perform one or more aspects of methods 100 and/or 200 again to recalibrate the patient's smartphone and/or reconfigure the sound generation parameters for the auditory biomarker detection test.

In some embodiments, method 300 alternatively implements block 312 between blocks 302 and 304 such that the application displays the prompt on the user interface with “Yes” and “No” (or similar) icons during the time while method 300 is implementing the above-described loop traversing blocks 304, 308, 310, 312, and 314. In some embodiments, the prompt can instead display a “Right” and “Left” for the patient to indicate which ear the patient heard the stimulus sound, whereupon activating the “Right” or “Left” icon indicates to the application that the patient heard the stimulus sound via the patient's right or left ear, respectively, and whereupon not activating either the “Right” or “Left” icon indicates to the application that the patient did not hear the stimulus sound via either the patient's right or left ear, respectively.

Under any of the implementations of block 312, if the application receives an indication from the patient at block 312 that the patient heard the stimulus sound (e.g., a “Yes” input via the graphical user interface), then method 300 advances to point 318, which is the end of method 300 and the start of method 400. In some embodiments, method 300 advances to point 318 only after the application receives an indication from the patient at block 312 that the patient heard the stimulus sound in both the right and left ears. For example, in such embodiments, if the patient confirms hearing the stimulus sound in the right ear but not the left ear, then method 300 continues in a loop-wise, iterative fashion traversing blocks 304, 308, 310, and 312 making adjustments to the stimulus sound played via the left headphone until the patient confirms hearing the stimulus sound via his or her left ear. Then, after the patient has confirmed hearing the stimulus sound in both the right and left ears, method 300 advances to point 318, which is the end of method 300 and the start of method 400.

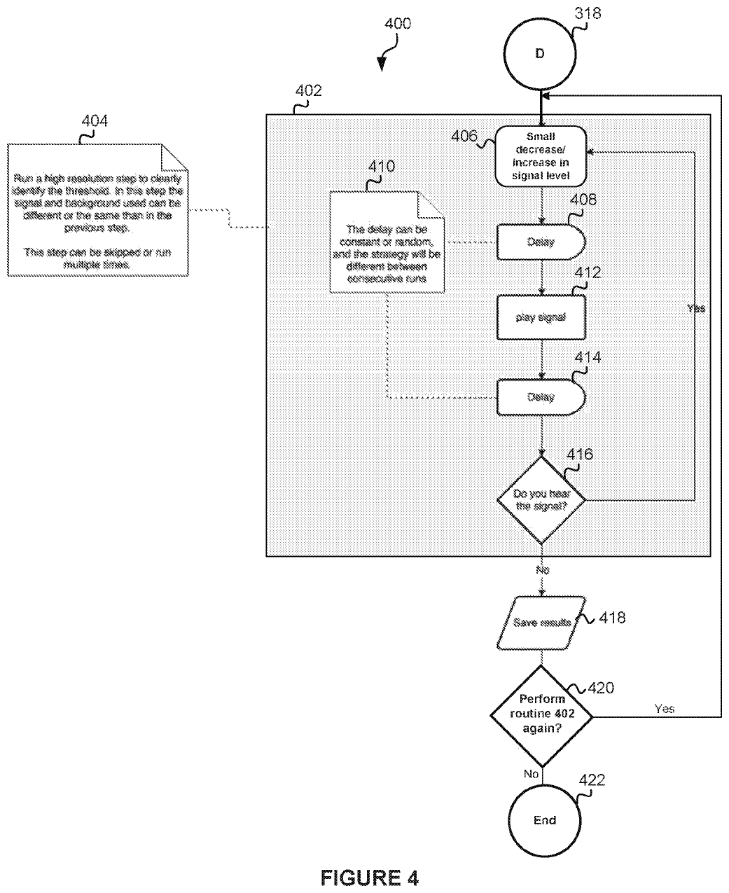

FIG. 4 shows a fourth method 400 employed in example embodiments of the systems and methods for detecting auditory biomarkers disclosed herein. Method 400 starts at point 318, at the conclusion of method 300.

Method 400 includes a high-resolution routine 402 that implements steps that are similar to the steps of method 300. Routine 402 is optional and need not be implemented, but if implemented, it can be run multiple times iteratively, as summarized in comment block 404. The goal of routine 402 is to better identify the lowest volume level at which the patient can hear the stimulus sound, as summarized in comment block 404. In operation, the application continues to play the background sound during the duration of routine 402.

In embodiments that do not implement routine 402, method 400 advances to block 418, where the application saves the results of method 300 into memory. In some embodiments, the results of method 300 include information about background and stimulus sounds used during method 300, e.g., the specific sounds, frequency, duration, intensities, and/or other data characterizing the background and stimulus sounds, and for each stimulus sound, whether the patient reporting hearing the sound or not.

In embodiments that implement routine 402 of method 400, routine 402 begins at block 406, where the application slightly (e.g., +/−1 dB) increases or decreases the volume setting for the stimulus sound from method 300 that the patient indicated he or she heard over the background sound at block 312 of method 300. In some embodiments, routine 402 uses the same background sound and same stimulus sound that the application used in method 300. In some embodiments, routine 402 uses a different background sound and/or a different stimulus sound that the application used in method 300. In some embodiments, at block 406, the application can additionally or alternatively alter one or more of frequency, duration, equalization, or other settings of the stimulus sound. In some embodiments, the degree to which the application increments or decrements the stimulus sound intensity during successive iterations of routine 402 is randomized up and down to approach, in an unpredictable way, the threshold level at which the patient can discern the stimulus from the background.

After slightly increasing or decreasing the volume setting (or perhaps otherwise altering) the stimulus sound at block 406, and while continuing to play the background sound, routine 402 advances to block 408, where a first delay is implemented before advancing to block 412, where the application plays the stimulus sound at the slightly altered volume setting from block 406. In some embodiments, the application plays the stimulus sound at block 412 for a fixed or random duration of time.

After playing the stimulus sound at block 412, and while continuing to play the background sound, routine 402 advances to block 414, where a second delay is implemented before advancing to block 416. In some embodiments, one or both of the first delay 408 and the second delay 414 can be a fixed or random duration of time. Similarly, the first delay implemented at block 408 and the second delay implemented at block 414 can be the same duration of time or different durations of time that are fixed or random, as summarized in comment block 410.

After implementing the second delay implemented at block 414, and while continuing to the play the background sound, routine 402 advances to block 416, where the application asks the patient whether the patient heard the stimulus sound. For example, the application can generate and display a prompt on the user interface with “Yes” and “No” (or similar) icons for the patient to select based on whether the patient heard the stimulus sound.

If the application receives an indication from the patient that the patient heard the stimulus sound (e.g., a “Yes” input via the graphical user interface), then routine 402 returns to block 406, where the routine 402 again modifies the volume setting of the stimulus sound, where modifying the volume of the stimulus sound includes incrementing or decrementing the volume, as shown in comment block 410.

After modifying the volume setting of the stimulus sound at block 406, and while continuing to play the background sound, routine 402 advances to block 408, where the application implements a first delay (which could be the same or a different duration as the previous time when the application implemented the first delay at block 408). And after expiration of the first delay at block 408, routine 402 advances to block 412, where the application plays the stimulus sound again, but at the modified volume level set at block 406. In some embodiments, the application plays the stimulus sound at block 412 again for a fixed or random duration of time, which can be the same or a different duration than the previous time the application played the stimulus sound at block 412 during execution of routine 402.

After playing the stimulus sound at block 412, routine 402 advances to block 414, where the application implements the second delay (which could be the same or a different duration as the previous time when the application implemented the second delay at block 414).

After expiration of the second delay at block 414, routine 402 advances to block 416, where the application again asks the patient whether the patient heard the stimulus sound. For example, the application can again generate and display a prompt on the user interface with “Yes” and “No” (or similar) icons for the patient to select based on whether the patient heard the stimulus sound.

If the application receives an indication from the patient that the patient heard the stimulus sound again (e.g., another “Yes” input via the graphical user interface), then routine 402 returns to block 406, and routine 402 continues in a loop-wise, iterative fashion traversing blocks 406, 408, 412, 414, and 416 until either: (i) the application receives an indication from the patient that the patient did not hear the stimulus sound (e.g., a “No” input via the graphical user interface) or (ii) the application receives a certain quantity of Yes indications from the patient that the patient heard the stimulus sound (e.g., a “Yes” input via the graphical user interface). For example, in some embodiments, if after a few iterations (e.g., between about 7-15 iterations) the patient continues to hear the stimulus sound, then routine 402 will stop and perhaps instruct the patient to perform one or more aspects of methods 100, 200, and/or 300 again to recalibrate the patient's smartphone according method 100, reconfigure the sound generation parameters for the auditory biomarker detection test according to method 200, and/or perform the initial auditory biomarker detection test again according to method 300.

In some embodiments, routine 402 alternatively implements block 416 between blocks 406 and 414 such that the application displays the prompt on the user interface with “Yes” and “No” (or similar) icons during the time while routine 402 is implementing the above-described loop traversing blocks 406, 408, 412, and 414. In some embodiments, the prompt can instead display a “Right” and “Left” for the patient to indicate which ear the patient heard the stimulus sound, whereupon activating the “Right” or “Left” icon indicates to the application that the patient heard the stimulus sound via the patient's right or left ear, respectively, and whereupon not activating either the “Right” or “Left” icon indicates to the application that the patient did not hear the stimulus sound on either the patient's right or left ear, respectively.

Under any of the implementations of block 416, when the application receives an indication from the patient at block 416 that the patient heard or did not hear the stimulus sound (e.g., a “Yes” or “No” input via the graphical user interface), then routine 402 advances to block 418, where the application stores the results of routine 402 and perhaps also the results of method 300 if the application has not previously done so. In some embodiments, the results of routine 402 include information about the background and stimulus sounds used during routine 402, e.g., the specific sounds, frequency, duration, intensities, other data characterizing the background and stimulus sounds, and for each stimulus sound, whether the patient reporting hearing the sound or not.

After saving the results at block 418, routine 402 advances to block 420, where the application again asks the patient whether he or she wishes to perform routine 402 again. In some embodiments, rather than asking the patient if he or she wishes to perform routine 402 again, the application could be configured instead to run routine 402 some number of times (e.g., perhaps 3-7 times) to obtain more results. Alternatively, in some embodiments, the application can be configured to only run routine 402 some limited number of times in a single day, e.g., about 1 or 3 times or perhaps only once per day, and in such embodiments, the application does not ask the patient whether he or she wishes to run routine 402 again.

If at block 420, routine 402 is to be run again either because the patient responds to a user interface prompt and confirms that he or she wishes to run routine 402 again or the application is configured to automatically run routine 402 at least one more time, the method 400 returns to block 406 where the application runs routine 402 in the loop-wise, iterative fashion described above.

But if at block 420, routine 402 is not to be run again because the patient responds to the user interface prompt and indicates that he or she does not wish to perform routine 402 again, the application has already automatically run routine 402 its configured number of times, or the application is configured to run routine only once per day, then method 400 advances to point 422, where method 400 ends.

As described above, one or more routines of an application can be implemented to determine auditory biomarkers for patients that experience migraine symptoms. For example, methods 100, 200, 300, and 400 allow for testing patients and thereby determining whether a given patient is likely to experience a migraine within a particular timeframe. Testing a population of patients in this manner can reveal statistical associations (e.g., correlations) between one or more of (i) auditory sensitivity, (ii) discrimination against background noise, and (iii) noise tolerance levels of patients, and experiencing migraine symptoms within a particular timeframe. For example, an increase in a threshold volume for discriminating a test signal from background noise for a given patient can indicate that a migraine is more likely to occur within a particular timeframe (e.g., 48 hours before a headache begins). As another example, an increase or decrease in a threshold volume level for a given patient can also indicate that a migraine is more likely to occur within the particular timeframe. As yet another example, a decrease in the patient's maximum comfort level can also indicate that a migraine is more likely to occur within the particular timeframe. Because the timing of such changes in how test signals are perceived can be different for each individual patient, a profile for each patient can be generated that tracks changes in how test signals are perceived. After (i) determining the profile for a given patient and (ii) receiving inputs via the application that match the profile, the application can provide an indication that a migraine attack is impending within a timeframe associated with the given patient, and possibly recommend a type of intervention for mitigating the migraine. This can allow time for mitigating treatment to be administered in advance of the migraine attack (e.g., acute medication, use of a device, therapeutic treatment, etc.).

Providing an indication that a migraine attack is impending can be based on a threshold change in hearing sensitivity in a given patient. For example, the application can first determine that a pre-determined auditory sensitivity, discrimination against background noise, and/or noise tolerance levels of a given patient have changed by a threshold amount. For example, a difference of more than a standard deviation from a median auditory sensitivity, a median discrimination against background noise, and/or a median noise tolerance levels can indicate that a migraine attack is impending. These thresholds levels can be unique to different patients based on inputs provided by each patient over time. Further the thresholds and/or pre-determined levels can change for a given patient over time. Still further, different volume levels can be affected differently for each patient, such that one volume level (e.g., an auditory sensitivity level) can be more predictive of an impending migraine than other volume levels. Accordingly, each patient can have unique circumstances in which the application provides an indication of an impending migraine.

Aspects of methods 100, 200, 300, and 400 can be combined as part of a process for detecting auditory biomarkers for one or more patients and for predicting symptoms for a disease or disorder based on detecting these auditory biomarkers. Within examples, a method including steps from one or more of methods 100, 200, 300, and 400 includes providing, by an audio output device (e.g., headphones), a plurality of sounds at varying intensity levels. For example, this may be performed in accordance with block 110. Within examples, a method including steps from one or more of methods 100, 200, 300, and 400 includes receiving, via a user interface, an input indicative of a perceived volume level of the plurality of sounds as perceived by a user. For example, this may be performed in accordance with block 110. Within examples, a method including steps from one or more of methods 100, 200, 300, and 400 includes determining, based on the received input, a plurality of user volume levels, wherein the plurality of user volume levels comprises (i) a threshold audible volume for the user, (ii) a comfortable volume level for the user, and (iii) a maximum comfort volume level for the user, and wherein the plurality of user volume levels indicate a hearing sensitivity of the user. For example, this may be performed in accordance with block 110. Within examples, a method including steps from one or more of methods 100, 200, 300, and 400 includes providing, by the audio output device, a background sound. For example, this may be performed in accordance with block 302. Within examples, a method including steps from one or more of methods 100, 200, 300, and 400 includes, concurrently while providing the background sound, providing, by the audio output device, a stimulus sound. For example, this may be performed in accordance with block 308. Within examples, a method including steps from one or more of methods 100, 200, 300, and 400 includes, receiving an indication, via the user interface, from the user that the user perceived the stimulus sound. For example, this may be performed in accordance with block 312. Within examples, a method including steps from one or more of methods 100, 200, 300, and 400 includes determining a change in the hearing sensitivity of the user based on receiving the indication that the user perceived the stimulus sound, and predicting an onset of a migraine attack of the user based on determining the change in the hearing sensitivity of the user.

Within examples, determining the plurality of user volume levels includes providing a plurality of input prompts corresponding to the varying intensity levels for defining each of the user volume levels, and receiving responses to the plurality of input prompts.

Within examples, determining the plurality of user volume levels includes determining the plurality of user volume levels independently for a left ear and a right ear of the user.

Within examples, a method including steps from one or more of methods 100, 200, 300, and 400 further includes, prior to providing the background sound, selecting the background sound from a background database and selecting the stimulus sound from a stimulus database.

Within examples, a method including steps from one or more of methods 100, 200, 300, and 400 further includes, The method of claim 1, further includes, prior to providing the background sound, setting a background volume level and an audio stimulus level. Providing the background sound can include providing the background sound at the background volume level, and providing the stimulus sound can include providing the stimulus sound at the stimulus volume level. Within these examples, determining the change in the hearing sensitivity of the user can include determining the change in the hearing sensitivity of the user based on the background volume level and the stimulus volume level. Within these examples, setting the background volume level and the audio stimulus level can include setting the background volume level and the audio stimulus level based on the plurality of user volume levels.

Within examples, a method including steps from one or more of methods 100, 200, 300, and 400 further includes, prior to providing the stimulus sound, (i) determining that the user did not perceive an initial stimulus sound, and (ii) modifying a volume level of the stimulus sound based on the determination that the user did not perceive the initial stimulus sound, within these examples, providing the stimulus sound can include providing the stimulus sound at the modified volume level.

Within examples, a method including steps from one or more of methods 100, 200, 300, and 400 further includes, implementing a first delay between providing the background sound and providing the stimulus sound.

Within examples, providing the stimulus sound can include providing the stimulus sound for a random duration of time.

Within examples, the background sound can be one of a plurality of background sounds played to the user and the stimulus sound is one of a plurality of stimulus sounds played to the user. Within these examples, a method including steps from one or more of methods 100, 200, 300, and 400 can further include, tracking sounds, frequency, duration, intensities, and other data characterizing the plurality of background sounds and the plurality of stimulus sounds.

Within examples, the stimulus sound can be a last stimulus sound of a plurality of stimulus sounds. Within these examples, a method including steps from one or more of methods 100, 200, 300, and 400 can further include, prior to providing the last stimulus sound, successively providing stimulus sounds of the plurality of stimulus sounds, and changing a stimulus volume level for each stimulus sound provided to the user for the user, and determining, that the user did not perceive any of the stimulus sounds successively provided prior to the last stimulus sound.

Within examples, a method including steps from one or more of methods 100, 200, 300, and 400 can further include, correlating a plurality of user volume levels with instances of migraine attacks for the user. Within these example, predicting the onset of the migraine attack of the user can include predicting the onset of the migraine attack of the user based on correlating the plurality of user volume levels with instances of migraine attacks for the user.

Within examples, determining the change in the hearing sensitivity of the user based on receiving the indication that the user perceived the stimulus sound can include determining that the user perceived the stimulus sound at a different stimulus noise level than a previous stimulus noise level perceived by the user.

Within examples, determining the change in the hearing sensitivity of the user based on receiving the indication that the user perceived the stimulus sound can include determining that the hearing sensitivity of the user has changed by a threshold amount, and predicting the onset of the migraine attack of the user based on determining the change in the hearing sensitivity of the user, can include predicting the onset of the migraine attack based on determining that the hearing sensitivity of the user has changed by the threshold amount.

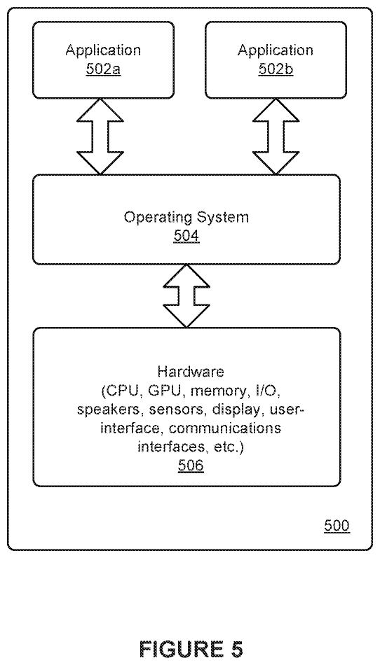

FIG. 5 shows an example computing device 500 configured to execute one or more (or all) of the features and functions of the auditory biomarker detection methods disclosed and described herein. The computing device 500 can be a smartphone, tablet, desktop or laptop computer, or any other type of computing device with the capability of generating and playing the background and stimulus sounds disclosed and described herein to a patient as well as performing any ancillary functions that can be required for effective implementation of the auditory biomarker detection methods disclosed and described herein.

Computing device 500 includes hardware 506 comprising: (i) one or more processors (e.g., a central processing unit(s) or CPU(s) and/or graphics processing unit(s) or GPU(s)); (ii) tangible non-transitory computer readable memory; (iii) input/output components (e.g., speaker(s), sensor(s), display(s), headphone jack(s) or other interfaces); and (iv) communications interfaces (wireless and/or wired). The hardware 506 components of the computing device 502 are configured to run software, including an operating system 504 (or similar) and one or more applications 502a, 502b (or similar) as is known in the computing arts. One or more of the applications 502a and 502b can correspond to computer-executable program code that, when executed by the one or more processors, cause the computing device 500 to perform one or more of the functions and features described herein, including but not limited to any (or all) of the features and functions of methods 100, 200, 300, and/or 400, as well as any other ancillary features and functions known to persons of ordinary skill in the computing arts that can be required or at least desired for effective implementation of the features and functions of methods 100, 200, 300, and/or 400, even if such ancillary features and/or functions are not expressly disclosed herein.

While particular aspects and embodiments are disclosed herein, other aspects and embodiments will be apparent to those skilled in the art in view of the foregoing teaching. For example, while the embodiments and examples are described with respect to migraine headaches, the disclosed systems and methods are not so limited and can be applicable to a broad range of disease symptoms and related disease factors and disease triggers. The various aspects and embodiments disclosed herein are for illustration purposes only and are not intended to be limiting, with the true scope being indicated by the following claims.

What is claimed is:

1. A method comprising:
providing, by an audio output device, a plurality of sounds at varying intensity levels;
receiving, via a user interface, an input indicative of a perceived volume level of the plurality of sounds as perceived by a user;
determining, based on the received input, a plurality of user volume levels, wherein the plurality of user volume levels comprises (i) a threshold audible volume for the user, (ii) a comfortable volume level for the user, and (iii) a maximum comfort volume level for the user, and wherein the plurality of user volume levels indicate a hearing sensitivity of the user;
providing, by the audio output device, a background sound;
concurrently while providing the background sound, providing, by the audio output device, a stimulus sound;
receiving an indication, via the user interface, from the user that the user perceived the stimulus sound;
determining a change in the hearing sensitivity of the user based on receiving the indication that the user perceived the stimulus sound; and
predicting an onset of a migraine attack of the user based on determining the change in the hearing sensitivity of the user.

2. The method of claim 1, wherein determining the plurality of user volume levels comprises:
providing a plurality of input prompts corresponding to the varying intensity levels for defining each of the user volume levels; and
receiving responses to the plurality of input prompts.

3. The method of claim 1, wherein determining the plurality of user volume levels comprises determining the plurality of user volume levels independently for a left ear and a right ear of the user.

4. The method of claim 1, further comprising:
prior to providing the background sound, selecting the background sound from a background database and selecting the stimulus sound from a stimulus database.

5. The method of claim 1, further comprising:
prior to providing the background sound, setting a background volume level and an audio stimulus level,
wherein providing the background sound comprises providing the background sound at the background volume level, and
wherein providing the stimulus sound comprises providing the stimulus sound at the stimulus volume level.

6. The method of claim 5, wherein determining the change in the hearing sensitivity of the user comprises determining the change in the hearing sensitivity of the user based on the background volume level and the stimulus volume level.

7. The method of claim 5, wherein setting the background volume level and the audio stimulus level comprises setting the background volume level and the audio stimulus level based on the plurality of user volume levels.

8. The method of claim 1, further comprising:
prior to providing the stimulus sound,
(i) determining that the user did not perceive an initial stimulus sound, and
(ii) modifying a volume level of the stimulus sound based on the determination that the user did not perceive the initial stimulus sound,
wherein providing the stimulus sound comprises providing the stimulus sound at the modified volume level.

9. The method of claim 1, further comprising:
implementing a first delay between providing the background sound and providing the stimulus sound.

10. The method of claim 1, wherein providing the stimulus sound comprises providing the stimulus sound for a random duration of time.

11. The method of claim 1, wherein the background sound is one of a plurality of background sounds played to the user and the stimulus sound is one of a plurality of stimulus sounds played to the user, the method further comprising:
tracking sounds, frequency, duration, intensities, and other data characterizing the plurality of background sounds and the plurality of stimulus sounds.

12. The method of claim 1, wherein the stimulus sound is a last stimulus sound of a plurality of stimulus sounds, the method further comprising:
prior to providing the last stimulus sound, successively providing stimulus sounds of the plurality of stimulus sounds, and changing a stimulus volume level for each stimulus sound provided to the user for the user; and
determining, that the user did not perceive any of the stimulus sounds successively provided prior to the last stimulus sound.

13. The method of claim 1, further comprising correlating a plurality of user volume levels with instances of migraine attacks for the user, wherein predicting the onset of the migraine attack of the user comprises predicting the onset of the migraine attack of the user based on correlating the plurality of user volume levels with instances of migraine attacks for the user.

14. The method of claim 1, wherein determining the change in the hearing sensitivity of the user based on receiving the indication that the user perceived the stimulus sound comprises determining that the user perceived the stimulus sound at a different stimulus noise level than a previous stimulus noise level perceived by the user.

15. The method of claim 1, wherein determining the change in the hearing sensitivity of the user based on receiving the indication that the user perceived the stimulus sound comprises determining that the hearing sensitivity of the user has changed by a threshold amount, and
wherein predicting the onset of the migraine attack of the user based on determining the change in the hearing sensitivity of the user comprises predicting the onset of the migraine attack based on determining that the hearing sensitivity of the user has changed by the threshold amount.

16. A non-transitory computer readable medium having computer-executable program code stored thereon that, when executed by one or more processors, causes performance of one or more functions, the functions comprising:
providing, by an audio output device, a plurality of sounds at varying intensity levels;
receiving, via a user interface, an input indicative of a perceived volume level of the plurality of sounds as perceived by a user;
determining, based on the received input, a plurality of user volume levels, wherein the plurality of user volume levels comprises (i) a threshold audible volume for the user, (ii) a comfortable volume level for the user, and (iii) a maximum comfort volume level for the user, and wherein the plurality of user volume levels indicate a hearing sensitivity of the user;
providing, by the audio output device, a background sound;
concurrently while providing the background sound, providing, by the audio output device, a stimulus sound;
receiving an indication, via the user interface, from the user that the user perceived the stimulus sound,
determining a change in the hearing sensitivity of the user based on receiving the indication that the user perceived the stimulus sound; and
predicting an onset of a migraine attack of the user based on determining the change in the hearing sensitivity of the user.

17. The non-transitory computer readable medium of claim 16, the functions further comprising:
prior to providing the stimulus sound,
(i) determining that the user did not perceive an initial stimulus sound, and
(ii) modifying a volume level of the stimulus sound based on the determination that the user did not perceive the initial stimulus sound,
wherein providing the stimulus sound comprises providing the stimulus sound at the modified volume level.

18. The non-transitory computer readable medium of claim 16, the functions further comprising:
correlating a plurality of user volume levels with instances of migraine attacks for the user, wherein predicting the onset of the migraine attack of the user comprises predicting the onset of the migraine attack of the user based on correlating the plurality of user volume levels with instances of migraine attacks for the user.

19. The non-transitory computer readable medium of claim 16, wherein determining the change in the hearing sensitivity of the user based on receiving the indication that the user perceived the stimulus sound comprises determining that the user perceived the stimulus sound at a different stimulus noise level than a previous stimulus noise level perceived by the user.

20. The non-transitory computer readable medium of claim 16, wherein determining the change in the hearing sensitivity of the user based on receiving the indication that the user perceived the stimulus sound comprises determining that the hearing sensitivity of the user has changed by a threshold amount, and
wherein predicting the onset of the migraine attack of the user based on determining the change in the hearing sensitivity of the user comprises predicting the onset of the migraine attack based on determining that the hearing sensitivity of the user has changed by the threshold amount.
